(19) United States
(12) Patent Application Publication
(10) Pub. No.: US 2026/0318912 A1
(43) Pub. Date: Oct. 15, 2026

(54) DETECTION ZONE OF A LATERAL FLOW IMMUNOASSAY DEVICE COMPRISING A POLYION MULTILAYER

(71) Applicant: Roche Diagnostics Operations, Inc., Indianapolis, IN (US)

(72) Inventors: Dan STROHMAIER-NGUYEN, Mannheim (DE); Carina HORN, Biblis (DE); Antje BAEUMNER, Regensburg (DE)

(21) Appl. No.: 19/682,949

(22) Filed: May 20, 2026

Related U.S. Application Data
(63) Continuation of application No. PCT/EP2024/082830, filed on Nov. 19, 2024.

(30) Foreign Application Priority Data
Nov. 20, 2023 (EP) 23210919.9

(51) Int. Cl. G01N 33/543

(52) U.S. Cl. CPC G01N 33/54388

(57) ABSTRACT

A first aspect of the invention is directed to a detection zone of a lateral flow immunoassay device comprising a polyion multilayer, the polyion multilayer comprising (i) at least one layer comprising a complex of at least a first polyion and a first member of a binding pair; and (ii) at least one layer comprising a second polyion, which is oppositely charged with respect to the first polyion. Lateral flow devices, kits, and methods of using the same are also provided.

### CROSS-REFERENCE TO RELATED APPLICATIONS

This application is a continuation of International Patent Application No. PCT/EP2024/082830, filed Nov. 19, 2024, which claims the benefit of priority to European Patent Application No. 23210919.9, filed Nov. 20, 2023, the disclosures of which are herein incorporated by reference in their entirety.

### TECHNICAL FIELD

The present disclosure generally relates to the field of immunoassays, e.g., lateral flow immunoassay devices.

### BACKGROUND

Recently, with the continuous process in point-of-care testing (POCT), fast and ultra-sensitive detection is accomplished by lateral flow immunoassays (LFAs). The simple, affordable, and user-friendly setup makes the LFA a relevant and efficient diagnostic tool where high-tech infrastructure may not be met. The fundamental part of the LFA is the porous membrane, for example, made of nitrocellulose, that enables passive sample migration through capillary forces and simple immobilization of proteins necessary for detection such as antibodies and streptavidin. However, the LFA also bears some major drawbacks and limitations such as variation of flow rate and analysis time due to varying pore structure and sample viscosity, obstruction of pores by matrix components, reasonably high sample volume, and inconsistency in the dispersion of the labeled sample to the membrane due to batch-to-batch variations of the membrane. In addition, as the LFA performance strongly depends on the properties of the membrane material, a change of the production line by the supplier results in changes in the membrane properties due to modifications of production parameters such as drying temperature and line speed, requiring re-optimization of an already finalized assay. Inter alia, since the membrane is used as a chromatography matrix, sample separation has to be realized by a relatively thick separation fleece, this in turn requiring a relatively large amount of sample in order to perform a measurement. For example, in case of blood samples, such large amounts of blood must be taken venously and cannot be taken at the fingertip, as such large amounts of blood sampling at the fingertip are very painful.

### BRIEF SUMMARY

The following passages introduce a selection of concepts in a simplified form that are further described below in the Detailed Description. This summary of selected aspects is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

If no membrane material is used within an LFA concept, immobilization of biorecognition elements in test and control lines must be re-evaluated. Several polymers such as polyethylene terephthalate (PET), poly(methyl methacrylate) (PMMA) and cyclic olefin copolymer (COC) have been used in biosensors due to their low cost with planar (2D) or three dimensional (3D) immobilization strategies. 2D approaches typically employ adsorption or covalent immobilization where the density and amount of the biorecognition molecules is limited to the active sites on the polymer surface itself. In some instances, the polymers' hydrophobicity can lead to partial denaturing of proteins so that tethers or spacers need to be employed. In contrast, 3D immobilization in a polymer matrix such as a hydrogel by encapsulation, copolymerization, electrostatic capture or covalent linking enables higher immobilization rates within a protective protein surrounding. The intrinsic swelling behavior of the hydrogel however requires an additional washing step to prevent non-specific signals and the overall chemistry involved is more complex than for simple adsorptive strategies.

Therefore, in some aspects, the present disclosure provides an assay device, which overcomes the drawbacks indicated above, i.e. an assay device which is especially free of a membrane.

### BRIEF DESCRIPTION OF THE DRAWINGS

Various features and advantages of aspects of the present disclosure will be more fully understood from the following detailed description of illustrative embodiments taken in conjunction with the accompanying drawings in which:

FIG. 1 shows characterization of the PDADMAC:PSA particles and the effect of the PDADMAC:PSA ratio on the size (a) and on the zeta potential of the PDADMAC:PSA complex (b). Error bars represent mean values+1σ and were calculated based on three parallel measurements of PDADMAC:PSA complexes (n=3)

FIG. 2 shows optimization of PDADMAC:PSA ratio and pH in complexation process. Plot of fluorescence intensity of a constant Rhodamine 6G concentration (0.05 mg*mL⁻¹) against the PDADMAC:PSA ratio (a). Plot of the fluorescence intensity of a constant Rhodamine 6G concentration (0.05 mg*mL⁻¹) against the pH in the complexation process. Error bars represent mean values+1σ and were calculated based on three parallel measurements of PDADMAC:PSA complexes (n=3).

FIG. 3 shows a plot of fluorescence intensity of the bioassay using a constant AG concentration of 1 ng·mL⁻¹ in buffer against the number of layer-by-layer cycles (a). Plot of the fluorescence intensity of the bioassay using a constant AG concentration of 1 ng·mL⁻¹ in buffer against the flow rate (b). Error bars represent mean values+1σ and were calculated based on three parallel measurements on three different LFAs (n=3).

FIG. 4 shows a lot of fluorescence intensity against logarithm of antigen concentration in spiked HBS buffer (a) and in spiked human serum samples (b) with logistic fit (red line) and corresponding parameters. Standard deviation were calculated based on 6 parallel measurements on 6 different LFAs, while outliers were removed after Q-test (confidence interval 95%). Error bars represent mean values±1σ (n≥4).

FIG. 5 shows the stability of the fluorescence signal of the bioassay using a constant AG concentration of 1 ng·mL⁻¹ over 8 weeks. The LFA were stored in an airtight capsule with a drying agent at 4° C. (n=3).

### DETAILED DESCRIPTION

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

### 1st Aspect—Detection Zone of a Lateral Flow Immunoassay Device

A first aspect of the invention thus relates to a detection zone of a lateral flow immunoassay device comprising a polyion multilayer, the polyion multilayer comprising
(i) at least one layer comprising a complex of at least a first polyion and a first member of a binding pair; and
(ii) at least one layer comprising a second polyion, which is oppositely charged with respect to the first polyion.

The detection zone of a lateral flow immunoassay device comprising a polyion multilayer enables a membrane-free concept for a LFA, which in turn enables short detection times and allows to use only very small sample values—in case of a blood analysis only about 20 μL sample volume are required, which allows for a convenient finger-prick sampling instead of taking blood from the vein. The resulting LFA not only benefits from easy assembly and low production costs but also demonstrated high sensitivity and overcomes the common drawbacks of traditional LFAs, i.e. need of a porous membrane and high sample volumes. The developed polyion multilayer design allows the usage significantly reduced volume of sample when compared to the conventional membrane-containing LFA format to achieve the same assay performance.

As used herein, the term “detection zone”, often also referred to as a measurement zone, refers to a region within a lateral flow immunoassay device where a detection of an analyte occurs. The detection may be analyte specific. The detection may be a qualitative and/or a quantitative detection and may further be electrochemical and/or optical. The term “electrochemical detection” refers to a direct or indirect (e.g. by using electrochemical mediators) detection of an electrochemically detectable property of the analyte, such as an electrochemical detection reaction. The term “optical detection” refers to a detection of an optical detectable property of the analyte itself or an auxiliary compound which is produced or converted within a detection reaction depending on the presence and/or concentration of the analyte in the sample, such as a color change and/or a change in remissive properties.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s).

The method as referred to in accordance with the present invention includes a method which essentially consists of the aforementioned steps or a method which includes further steps. However, it is to be understood that the method, in a preferred embodiment, is a method carried out ex vivo, i.e. not practiced on the human or animal body.

In some embodiments of the detection zone of a lateral flow immunoassay device, the first polyion of (i) and the second polyion of (ii) are selected from the group consisting of acrylamide copolymer, alginate, lignosulfonate, pectin, polyacrylic acid, co-polymer of polyacrylic acid, polyvinylsulfuric acid, polycarboxylic acid, polyphosphoric acid, polysaccharide, crosslinked polystyrene sulfonic acid, uncrosslinked polystyrene sulfonate, poly-diallyldialkyl-ammonium, polyethyleneimine, polyvinylamine, polyvinylpyridine, polyvinylammonium, partial or complete salts of these polyions, wherein the counter ions are selected from alkali cations, halogenide anions, and sulfonate, and mixtures of two or more of these polyions and/or their partial or complete salts; with the condition that first and second polyion are selected so that they have opposite charges.

The term “alkyl”, as used herein, relates to a straight or branched chain, saturated hydrocarbon group, linked to the main chain by a covalent bond to at least one of its at least one carbon atoms. Preferred alkyl groups are straight chain alkyls, e.g., preferably, methyl, ethyl, propyl, butyl, pentyl, hexyl, or branched chain C3 to C10 alkyl groups. Accordingly, alkyl groups include primary alkyl groups, secondary alkyl groups, and tertiary alkyl groups. An “alkali cation” is a cation of an element from the 1st group of the periodic system of the elements (PSE), preferably a sodium and/or a potassium cation. A “halogenide ion” is an anion of an element from the 17^{th} group of the PSE, preferably chloride or bromide, more preferably chloride.

In some embodiments of the detection zone of a lateral flow immunoassay device, the first polyion of (i) and the second polyion of (ii) are selected from the group consisting of poly(styrenesulfonate) (PSS), poly(vinyl sulfonic acid, (PVS), polyacrylic acid (PAA), poly(acrylamide-2-methylpropane sulfonate) (PAMPS), dextran sulfate, polyethylenimine (PEI), poly(diallyldimethylammonium) (PDADMAC), poly(vinylamine) (PVA), polyallylamine (PAH), poly(vinylbenzyltrimethyl ammonium) (PVTA), poly(N,N-dimethyl-3,5-dimethylene piperidinium) (PDDP), poly-L-lysine (PLL), chitosan, and mixtures of two or more thereof, with the condition that first and second polyion are selected so that they have opposite charges.

In some preferred embodiments of the detection zone of a lateral flow immunoassay device, the first polyion of (i) comprises, more preferably is, one or more polycation(s). Preferably, the one or more polycation(s) of (i) are selected from the group consisting of PEI, PDADMAC, PAH, PLL, chitosan, and mixtures of two or more thereof.

In some preferred embodiments of the detection zone of a lateral flow immunoassay device, the polycation of (i) comprises, preferably is PDADMAC. Preferably, the PDADMAC has in the range of from 619 to 2165 repetitive units (degree of polymerization).

In some preferred embodiments of the detection zone of a lateral flow immunoassay device, the second polyion of (ii) comprises, more preferably is, one or more polyanion(s). Preferably, the one or more polyanion(s) of (ii) are selected from the group consisting of PSS, PVS, PAA, dextran sulfate, and mixtures of two or more thereof. Preferably, the polyanion of (ii) comprises, more preferably is, PAA. Preferably, PAA has in the range of from 21 to 1388 repetitive units (degree of polymerization).

A “binding pair” as used herein consists of two partners binding to each other with high affinity, i.e. with one nanomolar affinity or better. In some preferred embodiments of the detection zone of a lateral flow immunoassay device, the first member of a binding pair is selected from the group consisting of antibody, nanoparticle functionalized with an antibody, nanoparticle functionalized with streptavidin, enzyme (such as glucoseoxidase), (poly)streptavidin, biotin, complementary nucleic acid (such as L-LNA, aptamer), protein A, protein G, molecular imprinted polymer (MIP), and mixtures of two or more thereof.

The term “antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. Preferably, the antibody is a polyclonal antibody. More preferably, the antibody is a monoclonal antibody.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The subunit structures and three-dimensional configurations of different classes of immunoglobulins are well known and described generally in, for example, Abbas et al., Cellular and Mol. Immunology, 4th ed., W.B. Saunders, Co. (2000). An antibody may be part of a larger fusion molecule, formed by covalent or non-covalent association of the antibody with one or more other proteins or peptides.

The term “antibody” means a “full-length antibody,” which interchangeably can be named “intact antibody,” or “whole antibody”, meaning an antibody in its substantially intact form, not antibody fragments as defined below. The terms particularly refer to an antibody with heavy chains that contain an Fc region. “Antibody fragments” comprise a portion of an intact antibody, in an embodiment, comprising the antigen-binding region thereof. Examples of antibody fragments include F(ab), F(ab′), F(ab′)2, and Fv fragments; diabodies; linear antibodies; single-chain antibody molecules; and multispecific antibodies formed from antibody fragments. Papain digestion of antibodies produces two identical antigen-binding fragments, called “F(ab)” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. “Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three hypervariable regions (HVRs) of each variable domain interact to define an antigen-binding site. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site. The term “diabodies” refers to antibody fragments with two antigen-binding sites, which fragments comprise a heavy-chain variable domain (VH) connected to a light-chain variable domain (VL) in the same polypeptide chain (VH-VL).

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds an analyte, wherein the analyte-binding polypeptide sequence was obtained by a process that includes the selection of a single analyte binding polypeptide sequence from a plurality of polypeptide sequences. For example, the selection process can be the selection of a unique clone from a plurality of clones, such as a pool of hybridoma clones, phage clones, or recombinant DNA clones. In contrast to polyclonal antibody preparations, which include different antibodies directed against different determinants (epitopes), each monoclonal antibody of a monoclonal-antibody preparation is directed against a single determinant on an antigen. In addition to their specificity, monoclonal-antibody preparations are advantageous in that they are typically uncontaminated by other immunoglobulins.

As used herein, the term “nanoparticle” refers to a particle being less than 100 nanometers in at least one dimension, i.e. having a diameter of less than 100 nm-determinable, for example, by TEM-measurements. Nanoparticles can be made from a variety of polymers, such as polystyrene (latex), polyacrylnitrile, or can be metal containing or metal based, wherein the metal is for example, gold (Au) or silver (Ag).

“LNA” means locked nucleic acid and “L-LNA” means beta-L-LNA single strand, i.e. the L-configured stereoisomer of LNA (see, for example, WO 2019/243391 A1, WO 2020/245377 A1).

An “aptamer” is a macromolecule specifically binding its interaction partner. Aptamers can be peptide or polynucleotide aptamers and are in principle known to the skilled person.

“Molecular imprinted polymers” (MIP) are polymers that mimic antibodies and/or receptors and are in principle known to the skilled person (see, for example, Ye L, Haupt K. Molecularly imprinted polymers as antibody and receptor mimics for assays, sensors and drug discovery. Anal Bioanal Chem. 2004 April; 378(8):1887-97).

“Protein A”, “protein G”, “streptavidin” and “(poly)streptavidin” as well as “biotin” are also well known to the skilled person.

In some preferred embodiments of the detection zone of a lateral flow immunoassay device, the first member of a binding pair of (i) is streptavidin, more preferably polystreptavidin, more preferably negatively charged polystreptavidin. In some preferred embodiments, then the corresponding second member of the binding pair is biotin.

In some preferred embodiments of the detection zone of a lateral flow immunoassay device, the polycation of (i) comprises, preferably is PDADMAC, the polyanion of (ii) comprises, preferably is, PAA, and the first member of a binding pair of (i) is streptavidin, more preferably polystreptavidin, more preferably negatively charged polystreptavidin.

Preferably, the detection zone of a lateral flow immunoassay device is membrane-free. “Membrane-free” means that there is no membrane, especially no nitrocellulose membrane, and generally no paper, fleece or membrane or the like material present within the detection zone.

In some preferred embodiments of the detection zone of a lateral flow immunoassay device, the polyion multilayer is positioned on a surface of a carrier. Preferably, the surface of the carrier is pre-treated with oxygen plasma and/or corona, more preferably with oxygen plasma, and subsequently with an aqueous solution of one or more polyion(s), preferably one or more polyion(s) selected from the groups indicated above.

In some preferred embodiments of the detection zone of a lateral flow immunoassay device, the polycation of (i) comprises, preferably is PDADMAC, the polyanion of (ii) comprises, preferably is, PAA, and the first member of a binding pair of (i) is streptavidin, more preferably polystreptavidin, more preferably negatively charged polystreptavidin; and the polyion multilayer is positioned on a surface of a carrier, wherein the surface of the carrier preferably is (or has been) pre-treated with oxygen plasma and/or corona, more preferably with oxygen plasma.

In some embodiments of the detection zone of a lateral flow immunoassay device the layers in the polyion multilayer are arranged horizontally on top of each other with respect to the carrier. Preferably, a layer comprising the second polyion (ii) is horizontally positioned on top of a layer comprising the first polyion and the first member of a binding pair (i) in the polyion multilayer,

In some preferred embodiments of the detection zone of a lateral flow immunoassay device, the polyion multilayer comprising at least two layers (i) and at least two layers (ii), wherein the layers (i) and (ii) are horizontally positioned in an alternating sequence on top of each other. Preferably, the final layer on top comprises one or more polyanion(s). This is especially advantageous since this arrangement helps to avoid unspecific bindings.

In some preferred embodiments of the detection zone of a lateral flow immunoassay device, the polycation of (i) comprises, preferably is PDADMAC, the polyanion of (ii) comprises, preferably is, PAA, and the first member of a binding pair of (i) is streptavidin, more preferably polystreptavidin, more preferably negatively charged polystreptavidin; and the polyion multilayer is positioned on a surface of a carrier, wherein the surface of the carrier preferably is (or has been) pre-treated with oxygen plasma and/or corona, more preferably with oxygen plasma, wherein preferably the polyion multilayer comprises at least two layers (i) and at least two layers (ii), wherein the layers (i) and (ii) are horizontally positioned in an alternating sequence on top of each other—the final layer on top preferably being a layer comprising one or more polyanion(s).

In some embodiments of the detection zone of a lateral flow immunoassay device, the carrier is a foil, preferably selected from the group consisting of gold foil, silver foil, carbon foil and polymer foil, wherein the polymer is selected from the group consisting of polyester, polyvinyl chloride, poly(methyl methacrylate), polydimethylsiloxane and mixtures of two or more of these polymers, wherein the polymer foil is more preferably a polyester foil, more preferably a poly(ethylene terephthalate) (PET) foil.

In some preferred embodiments of the detection zone of a lateral flow immunoassay device, the polycation of (i) comprises, preferably is PDADMAC, the polyanion of (ii) comprises, preferably is, PAA, and the first member of a binding pair of (i) is streptavidin, more preferably polystreptavidin, more preferably negatively charged polystreptavidin; and the polyion multilayer is positioned on a surface of a carrier, which is a polyester foil, preferably a poly(ethylene terephthalate) (PET) foil, wherein the surface of the carrier preferably is (or has been) pre-treated with oxygen plasma and/or corona, more preferably with oxygen plasma, wherein preferably the polyion multilayer comprises at least two layers (i) and at least two layers (ii), wherein the layers (i) and (ii) are horizontally positioned in an alternating sequence on top of each other—the final layer on top preferably being a layer comprising one or more polyanion(s).

In some preferred embodiments of the detection zone of a lateral flow immunoassay device, the at least one layer comprising a first polyion and a first member of a binding pair of (i) is obtained or obtainable by application of one or more aqueous solution(s) of first polyion and/or first member of a binding pair to a surface of the carrier, preferably to a pre-treated surface of the carrier, wherein the aqueous solution(s) has/have a pH in the range of from 7 to 8, preferably in the range of from 7.2 to 7.6; and/or the at least one layer comprising a second polyion of (ii) is obtained or obtainable by application of an aqueous solution of the second polyion, wherein the aqueous solution has a pH in the range of from 4 to 5, preferably in the range of from 4.3 to 4.7.

“Application of one or more aqueous solution(s) of first polyion and/or first member of a binding pair” means that a separate solution of first polyion and a separate solution of first member of a binding pair are used, or that one or more solution(s) are used containing both, i.e. first polyion and first member of a binding pair.

### 2^{nd} Aspect—Lateral Flow Immunoassay Device

In a second aspect, the invention relates to a lateral flow immunoassay device comprising a capillary channel, wherein the capillary channel houses
(I) a detection zone as defined in the section related to the first aspect above;
(II) a first reagent zone, which comprises a labeled binding moiety; and
(III) a second reagent zone, which comprises a binding moiety carrying a second member of a binding pair;
wherein the binding moieties of (II) and of (III) are both capable of binding an analyte of interest, and the second member of a binding pair is capable of binding with the first member of a binding pair of the detection zone.

The term “device”, as used herein, relates to a system of means comprising at least the aforementioned means operatively linked to each other as to allow determination.

All details, embodiments and preferred embodiments disclosed above in the section related to the first aspect (the detection zone) apply also for the lateral flow immunoassay device of the second aspect.

In some embodiments of the lateral flow immunoassay device, first reagent zone of (II) and second reagent zone of (III) are spatially separated from each other or overlap with each other at least partially.

In some embodiments of the lateral flow immunoassay device, the label of the labeled binding moiety is a compound capable of making the presence of a molecule or complex comprising said label detectable, preferably having an electrically or electrochemically detectable property and/or an optically detectable property, wherein the label is more preferably a dye or a luminescent compound. Dyes may be fluorescent dyes, for example, selected from the group of fluorescein, coumarin, rhodamine, oxazine, resorufin, cyanine, squaraine and derivatives thereof. Luminescent compounds may be luminescent metal complexes such as ruthenium or europium complexes.

“Electrically or electrochemically detectable property” refers to a detection of an electrochemically detectable property of the analyte itself or an auxiliary compound (e.g. a redoxmediator), which is produced or converted within a detection reaction depending on the presence and/or concentration of the analyte in the sample. For example, the electrochemically detectable property may be detected by comparing one or more electrode potentials, such as an electrostatic potential of a working electrode with the electrostatic potential of one or more further electrodes such as a counter electrode or a reference electrode. The term “Optically detectable property” refers to a detection of an optical detectable property of the analyte itself or an auxiliary compound (e.g. a dye) which is produced or converted within a detection reaction depending on the presence and/or concentration of the analyte in the sample, such as a color change and/or a change in remissive properties. The measurement may be a qualitative and/or a quantitative measurement.

In some embodiments of the lateral flow immunoassay device, the binding moiety of the labeled binding moiety is a polypeptide or a fragment thereof, preferably an antibody or a F(ab) fragment. In some embodiments of the lateral flow immunoassay device, the binding moiety of the biotin carrying binding moiety is a polypeptide or a fragment thereof, preferably an antibody or a F(ab) fragment. In some preferred embodiments of the lateral flow immunoassay device, the binding moiety of the labeled binding moiety is non-identical to the binding moiety of the biotin carrying binding moiety.

In some embodiments of the lateral flow immunoassay device, said device further comprises an inlet opening configured to receive a sample of a body fluid; a vent opening configured to provide an air vent to the capillary channel; and at least one measuring device.

The lateral flow immunoassay device comprising a capillary channel of the second aspect is or can be used in a test system for analyzing a sample of a bodily fluid as disclosed in WO 2017/216339 A1, wherein the capillary channel housing the detection zone is comprised in a test strip. Preferably, the measuring device comprises at least one sealing element for hermetically sealing the vent opening of the test strip from an ambient atmosphere, at least one suction device adapted to provide an underpressure to the vent opening and optionally at least one valve configured to optionally vent the vent opening.

### 3^{rd} Aspect—Use

A third aspect of the present invention is related to the use of the lateral flow immunoassay device of the second aspect described above for determining an analyte in a sample. All details, embodiments and preferred embodiments disclosed in the sections related to the first aspect (detection zone) and the second aspect (lateral flow immunoassay device) apply as well for the third aspect.

In some preferred embodiments related to the use, the analyte is selected from the group consisting of protein, peptide, lipid, polysaccharide, antigen of an infection disease, and small molecule, preferably selected from the group consisting of a peptide, protein, and antigen of infection disease, more preferred selected from the group consisting of N-terminal pro-brain natriuretic peptide (NT-proBNP), cardiac troponin T (TnT), D-dimer, beta human chorionic gonadotropin (beta-HCG), Sars-COV-2 virus, influenza A (FluA) virus, influenza B (FLuB) virus, respiratory syncytial virus (RSV) and soluble form of triggering receptor expressed on myeloid cells 1 (sTREM1), more preferred N-terminal pro-brain natriuretic peptide (NT-proBNP). Preferably, the sample is a body fluid sample of a subject, more preferably selected from the group consisting of saliva, urine, nasal fluid, blood, plasma and serum sample of a subject, wherein the subject is preferably a mammal, more preferably a human.

### 4^{th} Aspect—Method for Determining an Analyte in a Sample

In a fourth aspect, the invention relates to a method for determining an analyte in a sample, the method comprising
(a) contacting a sample with at least a labeled binding moiety and a binding moiety carrying a second member of a binding pair, thereby forming a mixture;
(b) contacting the mixture formed in (a) with a polyion multilayer, the polyion multilayer comprising
   (i) at least one layer comprising a complex of at least a first polyion and a first member of a binding pair,
   (ii) at least one layer comprising a second polyion, thereby optionally forming complexes;
(c) determining the amount of label containing complexes formed in (b);
(d) determining said analyte in a sample based on the result of step (c).

“Optionally forming complexes” means that a complex, preferably a sandwich complex, is formed in case analyte of interest is present in the sample. “Sandwich assay” means detection by means of a non-competitive assay, requiring two different antibodies that recognize the antigen and do not interfere with each other's binding to the antigen. Complexes are formed between analyte, labeled binding moiety and binding moiety carrying a second member of a binding pair, wherein the sandwich complex is captured in the detection zone via binding between first and second member of a binding pair.

All details, embodiments and preferred embodiments disclosed in the sections related to the first aspect (detection zone) and the second aspect (lateral flow immunoassay device), as well as the third aspect (use), apply as well for the method of the fourth aspect.

Preferably, the second member of a binding pair is selected from the group consisting of antibody, nanoparticle functionalized with an antibody, nanoparticle functionalized with streptavidin, enzyme (such as glucoseoxidase), (poly)streptavidin, biotin, complementary nucleic acid (such as L-LNA, aptamer), protein A, protein G, molecular imprinted polymer (MIP), and mixtures of two or more thereof, with the condition that second member of a binding pair of (a) and first member of a binding pair of (i) are selected to that they are capable of binding with each other.

In some preferred embodiments of the method for determining an analyte in a sample, the analyte is selected from the group consisting of protein, peptide, lipid, polysaccharide, antigen of an infection disease, and small molecule, preferably selected from the group consisting of a peptide, protein, and antigen of infection disease, more preferred selected from the group consisting of N-terminal pro-brain natriuretic peptide (NT-proBNP), cardiac troponin T (TnT), D-dimer, beta human chorionic gonadotropin (beta-HCG), Sars-COV-2 virus, influenza A (FluA) virus, influenza B (FLuB) virus, respiratory syncytial virus (RSV) and soluble form of triggering receptor expressed on myeloid cells 1 (sTREM1), more preferred N-terminal pro-brain natriuretic peptide (NT-proBNP). Preferably, the sample is a body fluid sample of a subject, more preferably selected from the group consisting of saliva, urine, nasal fluid, blood, plasma and serum sample of a subject, wherein the subject is preferably a mammal, more preferably a human.

### 5^{th} Aspect—Kit

A fifth aspect of the invention relates to a kit for determining an analyte in a sample, comprising the lateral flow immunoassay device of the second aspect and a pump, which is connected or connectable to the suction device.

All details, embodiments and preferred embodiments disclosed in the sections related to the first aspect (detection zone), the second aspect (lateral flow immunoassay device), the third aspect (use), and the fourth aspect (method), apply as well for the kit of the fifth aspect.

Preferably, the kit further comprises an analysis device, which is configured to detect the label, preferably configured to detect an electrically or electrochemically detectable property and/or an optically detectable property, preferably an optically detectable property, of the label.

The present invention is further illustrated by the following set of embodiments and combinations of embodiments resulting from the dependencies and back-references as indicated. In particular, it is noted that in each instance where a range of embodiments is mentioned, for example in the context of a term such as “The method of any one of embodiments 1 to 4”, every embodiment in this range is meant to be explicitly disclosed for the skilled person, i.e. the wording of this term is to be understood by the skilled person as being synonymous to “The method of any one of embodiments 1, 2, 3 and 4”. Further, it is explicitly noted that the following set of embodiments represents a suitably structured part of the general description directed to preferred aspects of the present invention, and, thus, suitably supports, but does not represent the claims of the present invention.
Embodiment 1: A detection zone of a lateral flow immunoassay device comprising a polyion multilayer, the polyion multilayer comprising
(i) at least one layer comprising a complex of at least a first polyion and a first member of a binding pair; and
(ii) at least one layer comprising a second polyion, which is oppositely charged with respect to the first polyion.
Embodiment 2: The detection zone of a lateral flow immunoassay device of Embodiment 1, wherein the first polyion of (i) and the second polyion of (ii) are selected from the group consisting of acrylamide copolymer, alginate, lignosulfonate, pectin, polyacrylic acid, co-polymer of polyacrylic acid, polyvinylsulfuric acid, polycarboxylic acid, polyphosphoric acid, polysaccharide, crosslinked polystyrene sulfonic acid, uncrosslinked polystyrene sulfonate, poly-diallyldialkyl-ammonium, polyethyleneimine, polyvinylamine, polyvinylpyridine, polyvinylammonium, partial or complete salts of these polyions, wherein the counter ions are selected from alkali cations, halogenide anions, and sulfonate, and mixtures of two or more of these polyions and/or their partial or complete salts; with the condition that first and second polyion are selected so that they have opposite charges.
Embodiment 3: The detection zone of a lateral flow immunoassay device of Embodiment 1 or 2, wherein the first polyion of (i) and the second polyion of (ii) are selected from the group consisting of poly(styrenesulfonate) (PSS), poly(vinyl sulfonic acid, (PVS), polyacrylic acid (PAA), poly(acrylamide-2-methylpropane sulfonate) (PAMPS), dextran sulfate, polyethylenimine (PEI), poly(diallyldimethylammonium) (PDADMAC), poly(vinylamine) (PVA), polyallylamine (PAH), poly(vinylbenzyltrimethyl ammonium) (PVTA), poly(N,N-dimethyl-3,5-dimethylene piperidinium) (PDDP), poly-L-lysine (PLL), chitosan, and mixtures of two or more thereof, with the condition that first and second polyion are selected so that they have opposite charges.
Embodiment 4: The detection zone of a lateral flow immunoassay device of any one of Embodiments 1 to 3, wherein the first polyion of (i) comprises, preferably is, one or more polycation(s).
Embodiment 5: The detection zone of a lateral flow immunoassay device of Embodiment 4, wherein the one or more polycation(s) of (i) are selected from the group consisting of PEI, PDADMAC, PAH, PLL, chitosan, and mixtures of two or more thereof.
Embodiment 6: The detection zone of a lateral flow immunoassay device of Embodiment 4 or 5, wherein the polycation of (i) comprises, preferably is PDADMAC.
Embodiment 7: The detection zone of a lateral flow immunoassay device of any one of Embodiments 4 to 6, wherein the PDADMAC has in the range of from 619 to 2165 repetitive units (degree of polymerization).
Embodiment 8: The detection zone of a lateral flow immunoassay device of any one of Embodiments 1 to 7, wherein the second polyion of (ii) comprises, preferably is, one or more polyanion(s).
Embodiment 9: The detection zone of a lateral flow immunoassay device of Embodiment 8, wherein the one or more polyanion(s) of (ii) are selected from the group consisting of PSS, PVS, PAA, dextran sulfate, and mixtures of two or more thereof.
Embodiment 10: The detection zone of a lateral flow immunoassay device of Embodiment 8 or 9, wherein the polyanion of (ii) comprises, preferably is, PAA.
Embodiment 11: The detection zone of a lateral flow immunoassay device of any one of Embodiments 8 to 10, wherein the PAA has in the range of from 21 to 1388 repetitive units (degree of polymerization).
Embodiment 12: The detection zone of a lateral flow immunoassay device of any one of Embodiments 1 to 11, wherein the first member of a binding pair is selected from the group consisting of antibody, nanoparticle functionalized with an antibody, nanoparticle functionalized with streptavidin, enzyme (such as glucoseoxidase), (poly)streptavidin, biotin, complementary nucleic acid (such as L-LNA, aptamer), protein A, protein G, molecular imprinted polymer (MIP), and mixtures of two or more thereof.
Embodiment 13: The detection zone of a lateral flow immunoassay device of any one of Embodiments 1 to 12, wherein the first member of a binding pair of (i) is streptavidin, preferably polystreptavidin, more preferably negatively charged polystreptavidin.
Embodiment 14: The detection zone of a lateral flow immunoassay device of any one of Embodiments 1 to 13, being membrane-free.
Embodiment 15: The detection zone of a lateral flow immunoassay device of Embodiment 14, wherein the polyion multilayer is positioned on a surface of a carrier.
Embodiment 16: The detection zone of a lateral flow immunoassay device of Embodiment 15, wherein the surface of the carrier is pre-treated with oxygen plasma and/or corona, preferably with oxygen plasma, and subsequently with an aqueous solution of one or more polyion(s), preferably one or more polyion(s) selected from the groups indicated in any one of embodiments 2 to 11.
Embodiment 17: The detection zone of a lateral flow immunoassay device of any one of Embodiments 1 to 16, wherein in the polyion multilayer, the layers are arranged horizontally on top of each other with respect to the carrier.
Embodiment 18: The detection zone of a lateral flow immunoassay device of any one of Embodiments 1 to 17, wherein in the polyion multilayer, a layer comprising the second polyion (ii) is horizontally positioned on top of a layer comprising the first polyion and the first member of a binding pair (i).
Embodiment 19: The detection zone of a lateral flow immunoassay device of Embodiment 17 or 18, the polyion multilayer comprising at least two layers (i) and at least two layers (ii), wherein the layers (i) and (ii) are horizontally positioned in an alternating sequence on top of each other.
Embodiment 20: The detection zone of a lateral flow immunoassay device of any one of Embodiments 17 to 19, wherein the final layer on top comprises one or more polyanion(s).
Embodiment 21: The detection zone of a lateral flow immunoassay device of any one of Embodiments 14 to 20, wherein the carrier is a foil, preferably selected from the group consisting of gold foil, silver foil, carbon foil and polymer foil, wherein the polymer is selected from the group consisting of polyester, polyvinyl chloride, poly (methyl methacrylate), polydimethylsiloxane and mixtures of two or more of these polymers, wherein the polymer foil is more preferably a polyester foil, more preferably a poly(ethylene terephthalate) (PET) foil.
Embodiment 22: The detection zone of a lateral flow immunoassay device of any one of Embodiments 1 to 21, wherein the at least one layer comprising a first polyion and a first member of a binding pair of (i) is obtained or obtainable by application of one or more aqueous solution(s) of first polyion and/or first member of a binding pair to a surface of the carrier, preferably to a pre-treated surface of the carrier, wherein the aqueous solution(s) has/have a pH in the range of from 7 to 8, preferably in the range of from 7.2 to 7.6; and/or the at least one layer comprising a second polyion of (ii) is obtained or obtainable by application of an aqueous solution of the second polyion, wherein the aqueous solution has a pH in the range of from 4 to 5, preferably in the range of from 4.3 to 4.7.
Embodiment 23: A lateral flow immunoassay device comprising a capillary channel, wherein the capillary channel houses:
(I) a detection zone as defined in any one of Embodiments 1 to 22;
(II) a first reagent zone, which comprises a labeled binding moiety; and
(III) a second reagent zone, which comprises a binding moiety carrying a second member of a binding pair;
wherein the binding moieties of (II) and of (III) are both capable of binding an analyte of interest, and the second member of a binding pair is capable of binding with the first member of a binding pair of the detection zone.
Embodiment 24: The lateral flow immunoassay device of Embodiment 23, wherein first reagent zone of (II) and second reagent zone of (III) are spatially separated from each other or overlap with each other at least partially.
Embodiment 25: The lateral flow immunoassay device of Embodiment 23 or 24, wherein the label of the labeled binding moiety is a compound capable of making the presence of a molecule or complex comprising said label detectable, preferably having an electrically or electrochemically detectable property and/or an optically detectable property, wherein the label is more preferably a dye or a luminescent compound.
Embodiment 26: The lateral flow immunoassay device of any one of Embodiments 23 to 25, wherein the binding moiety of the labeled binding moiety is a polypeptide or a fragment thereof, preferably an antibody or a F(ab) fragment.
Embodiment 27: The lateral flow immunoassay device of any one of Embodiments 23 to 26, wherein the binding moiety of the biotin carrying binding moiety is a polypeptide or a fragment thereof, preferably an antibody or a F(ab) fragment.
Embodiment 28: The lateral flow immunoassay device of any one of Embodiments 23 to 27, wherein the binding moiety of the labeled binding moiety is non-identical to the binding moiety of the biotin carrying binding moiety.
Embodiment 29: The lateral flow immunoassay device of any one of Embodiments 23 to 29, further comprising an inlet opening configured to receive a sample of a body fluid; a vent opening configured to provide an air vent to the capillary channel; and at least one measuring device.
Embodiment 30: The lateral flow immunoassay device of Embodiment 29, wherein the measuring device comprises at least one sealing element for hermetically sealing the vent opening of the test strip from an ambient atmosphere, at least one suction device adapted to provide an underpressure to the vent opening and optionally at least one valve configured to optionally vent the vent opening.
Embodiment 31: Use of the lateral flow immunoassay device of any one of Embodiments 23 to 30 for determining an analyte in a sample.
Embodiment 32: A method for determining an analyte in a sample, the method comprising
(a) contacting a sample with at least a labeled binding moiety and a binding moiety carrying a second member of a binding pair, thereby forming a mixture;
(b) contacting the mixture formed in (a) with a polyion multilayer, the polyion multilayer comprising
   (i) at least one layer comprising a complex of at least a first polyion and a first member of a binding pair,
   (ii) at least one layer comprising a second polyion, thereby optionally forming complexes;
(c) determining the amount of label containing complexes formed in (b);
(d) determining said analyte in a sample based on the result of step (c).
Embodiment 33: The method of Embodiment 32, wherein the second member of a binding pair is selected from the group consisting of antibody, nanoparticle functionalized with an antibody, nanoparticle functionalized with streptavidin, enzyme (such as glucoseoxidase), (poly)streptavidin, biotin, complementary nucleic acid (such as L-LNA, aptamer), protein A, protein G, molecular imprinted polymer (MIP), and mixtures of two or more thereof, with the condition that second member of a binding pair of (a) and first member of a binding pair of (i) are selected to that they care capable of binding with each other
Embodiment 34: The use of Embodiment 31 or the method of Embodiment 32 or 33, wherein the analyte is selected from the group consisting of protein, peptide, lipid, polysaccharide, antigen of an infection disease, and small molecule, preferably selected from the group consisting of a peptide, protein, and antigen of infection disease, more preferred selected from the group consisting of N-terminal pro-brain natriuretic peptide (NT-proBNP), cardiac troponin T (TnT), D-dimer, beta human chorionic gonadotropin (beta-HCG), Sars-COV-2 virus, influenza A (FluA) virus, influenza B (FLuB) virus, respiratory syncytial virus (RSV) and soluble form of triggering receptor expressed on myeloid cells 1 (sTREM1), more preferred N-terminal pro-brain natriuretic peptide (NT-proBNP).
Embodiment 35: The use of Embodiment 31 or the method of Embodiment 32 or 33, wherein the sample is a body fluid sample of a subject, preferably selected from the group consisting of saliva, urine, nasal fluid, blood, plasma and serum sample of a subject, wherein the subject is preferably a mammal, more preferably a human.
Embodiment 36: A kit for determining an analyte in a sample, comprising the lateral flow immunoassay device of any one of Embodiments 23 to 30 and a pump, which is connected or connectable to the suction device.
Embodiment 37: The kit of Embodiment 36 further comprising an analysis device, which is configured to detect the label, preferably configured to detect an electrically or electrochemically detectable property and/or an optically detectable property, preferably an optically detectable property, of the label.

### EXAMPLES

The following Examples shall merely illustrate the invention. They shall not be construed whatsoever as limiting its scope.

### General Methods and Materials

The biotinylated capture antibody (polyclonal NT-proBNP sheep-IgG-biotin, cAb), antigen (NT-proBNP (1-76) amid) in buffer or human serum, probe antibody (monoclonal NT-proBNP mouse-IgG), probe antibody-modified fluorescence nanoparticles (Ab-fluorescence NPs), Albumin RPLA (97%), and polystreptavidin (PSA) were provided by Roche Diagnostics GmbH (Mannheim, Germany). Hydrochloric acid (HCl, 0.1 M, 1 M), sodium chloride (NaCl, p.a.), Bovine serum albumin (BSA, >96%), Poly(diallyldimethylammonium chloride) (PDADMAC, Mw 200,000-350,000, 20 wt. % in H₂O), Poly(acrylic acid, sodium salt) solution (average Mw 15,000, 35 wt. % in H₂O), Ethylenediaminetetraacetic acid (EDTA, ≥98.5%), Sodium hydroxide (NaOH, 1M), Poly(ethylene glycol)-block-poly(propylene glycol)-block-poly(ethylene glycol) (Synperonic® PE/P84), Sodium azide, Tween 20 (>97%) and Rhodamine 6 G (Rh-6G, 0.05 mg/mL) were supplied from Sigma-Aldrich (www.sigmaaldrich.com). Sucrose was purchased from Serva (www.serva.de). cobas h 232 NT-proBNP Plus Test was provided by Roche Diagnostics (Mannheim, Germany).

HEPES-buffered saline (HBS) consisted of 50 mM HEPES, 150 mM NaCl, 3 mM EDTA and 0.05% (w/v) Tween 20 and was adjusted to pH 7.4. HEPES dispensing buffer was prepared with 50 mM HEPES, 1% (w/v) Albumin RPLA, 1% (w/v) Sucrose, 0.15% (w/v) Synperonic PE/P84 and 0.024% (w/v) Sodium azide and was adjusted to pH 7.4. HEPES blocking buffer was prepared with 50 mM HEPES, 2.5% (w/v) Albumin RPLA, 0.15% (w/v) Synperonic PE/P84 and was adjusted to pH 7.4.

For particle characterization, the Zetasizer Ultra Pro (www.malvernpanalytical.com) was used. All drying procedures were done with the drying cabinet at 50° C. (FED 400 E2, www.binder-world.com). For plasma treatment, the plasma oven (www.gs-technologie.de) was used. All fluorescence images were acquired using a fluorescence microscope, LINOS lens (www.excelitas.com), HTC camera with a sony CCD sensor ICX285AL (www.sony.com), XENON XBO R 100W/45 OFR lamp (www.osram.com), 633 nm excitation filter and 685 nm detection filter (www.semrock.com) and a software provided by Roche Diagnostics (Mannheim, Germany). The fluorescence images were taken with an exposure time of 25 ms. Image processing and data analysis were carried out with ImageJ and Origin 2021.

### Example 1: Complex Formation Between Polydiallyldimethylammonium (PDADMA) and Polystreptavidin (PSA)

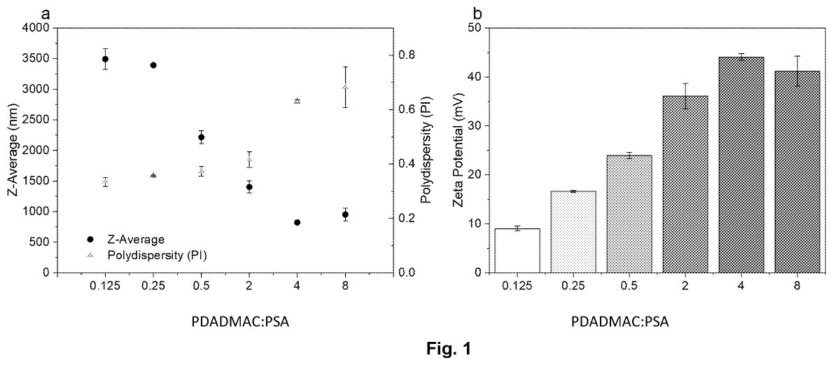

Cationic polydiallyldimethylammonium chloride (cationic PDADMAC) and negatively charged polystreptavidin (negatively charged PSA) were used to synthesize biofunctional microparticles. The characteristics of the polyelectrolyte—protein complex, especially the size and the charge of the complex depended on the nature of intensity of the polyelectrolyte protein interactions and on the environment. Thus, a crucial parameter influencing the complexation process was the ratio of the polyelectrolyte and protein used. Complexation of PDADMAC and PSA was performed by mixing both materials in different ratios at pH 7.4 and 150 mM NaCl. Directly after the mixing process, highly turbid samples were obtained immediately. Dynamic light scattering (DLS) analysis and zeta potential measurements proved the complexation process. The results are graphically shown in FIG. 1. The size and polydispersity index (PdI) of the resulting composite particles were dependent on the PDADMAC:PSA ratio (FIG. 1a). Specifically, when PSA was used in excess, a maximum of size could be found with a minimum of PdI, representing a tall mesh-like network where streptavidin bridged two or several PDADMA chains. This situation coexisted with free parts on the chain, i.e. without bound streptavidin, which provided the electrostatic repulsion to prevent aggregation. Thus, this state represented a network that is cross-linked by streptavidin. However, when PDADMAC was used in excess small particles were found with relatively high PdI values. This led to the assumption that the small amount of streptavidin was bound in a tightly compact way that is covered and governed by the PDADMAC. The high PdI could be explained by the fact that some parts of the PDADMAC sticked out at the surface, like a core corona structure. In case of the zeta potential of the resulting composite material, it was observed that it naturally became more positive with higher PDADMAC:PSA ratios, where likely above a ratio of 2 PDADMAC fully covered the particles (FIG. 1b).

### Example 2: Preparation of a Lateral Flow Immunoassay

### 2.1 Pretreatment of Substrate and Blocking of Non-Specific Adsorption

As base, a substrate having the form of a strip and having a length and width was used. First, the substrate (MELINEX®329, a film of biaxial-orientated polyethylene terephthalate (PET), thickness 175 μm) was pre-treated with oxygen plasma for 2 min, 100 W in order to increase the hydrophilicity and thus, enhancing the polyelectrolyte multilayer attachment. For blocking non-specific adsorption, a thin polyacrylic acid (PAA) layer was used on top. Therefore, the substrate was dip coated with PAA (0.2% (w/v), 150 mM NaCl, pH 7.4) for 60 seconds, washed with distilled water and dried at 50° C. The pre-treated substrate was then utilized for polyelectrolyte streptavidin multilayer deposition.

### 2.2 Polydiallyldimethylammonium-Polystreptavidin Particles (PPPs) Fabrication

For the PPP fabrication, a modified procedure of van der Straeten et al. (A. Vander Straeten et al., “Protein-polyelectrolyte complexes to improve the biological activity of proteins in layer-by-layer assemblies,” Nanoscale, vol. 9, no. 44, pp. 17186-17192, 2017, doi: 10.1039/C7NR04345G) was utilized. Polydiallyldimethylammonium-polystreptavidin particles (PPP) were fabricated by mixing polydiallyldimethylammonium chloride (PDADMAC) with polystreptavidin. First, a PDADMAC solution was prepared in 150 mM NaCl and polystreptavidin was dissolved in water to reach an end concentration of 0.1% (w/v) and 10 mg/mL, respectively, and both solutions were adjusted to pH 7.4. Then, 500 μL PDADMAC solution were mixed with 500 μL polystreptavidin solution to generate a solution containing the functionalized particles (PPP solution). The PPP solution was stored at 4° C. until further use.

### 2.2 Polyacrylic Acid Solution

Polyacrylic acid was dissolved in 150 mM NaCl to a concentration of 0.5% (w/v) and was adjusted to pH 4.55 to give polyacrylic acid solution (PAA solution).

### 2.3 Fluidic Assisted Polyelectrolyte Streptavidin Multilayer Lane Assembly and Antibody Dispensing

Polyelectrolyte streptavidin multilayer (PEM) lanes were deposited onto the substrate, i.e. the base of the microfluidic sensor, using a fluidic immobilization channel, which was arranged so that the channel's length was parallel to the substrate's width in a first end region of the pre-treated substrate—in the following, the “pre-treated substrate” is abbreviated as “the substrate”. To assemble the polyelectrolyte multilayer the PPP solution and PAA solution were alternate deposited onto the substrate by filling the immobilization channel by solely capillary forces. The fluidic deposition channel with double-sided adhesive tape was bonded onto the substrate and was used as a shaping lane mold. By using a stop flow approach, the channel was completely filled with the PPP solution and maintained there for 60 seconds. After the deposition time, the capillary was emptied and filled with the PAA solution and maintained there for 60 seconds. This alternate coating process was repeated for a given number of cycles for the preparation of the PEMs. Afterwards, the channel mold was relieved from the substrate and the polyelectrolyte streptavidin multilayer was dried at 50° C. for 60 seconds, thereby obtaining a PEM lane over the substrate's width in the first end region of the substrate.

In the next step, the antibodies were dispensed onto the substrate in a second end region of the substrate. The biotinylated human polyclonal anti-cardiac NT-proBNP capture antibody was prepared at a concentration of 300 μg/mL and the human specific monoclonal anti-cardiac NT-proBNP detection antibody-modified fluorescence nanoparticles was prepared at a concentration of 2% (w/v) in the HEPES dispensing buffer. 1.5 μL of each antibody were then dispensed onto the substrate in the second end region of the substrate at separate positions and were dried at 40° C. for 3 minutes.

### 2.4 Lateral Flow Immunoassay

The substrate with the streptavidin lane and probe antibodies of 2.3 was adhered to the spacer and cover foil and stored at 4° C. until further use.

A lateral flow immunoassay (LFA) was built up from four components:
1) the substrate (MELINEX®329, a PET film, thickness 175 μm) carrying thereon the PEM in a first end region of the substrate and the antibodies in a second end region of the substrate,
2) a spacer (MELINEX®329, PET film, thickness 250 μm) with laser cut capillaries,
3) a cover foil with sample port and outlet port and
4) pressure-sensitive double sided adhesive tape.

Substrate and spacer Melinex®329 (175 μm and 250 μm) were purchased from Dupont Teijin Films (www.dupontteijinfilms.com), the cover foil (Hostaphan RN 100) was purchased from Mitsubishi Polyester Film (www.m-petfilm.de) and double-sided adhesive tape was supplied from Henkel-Adhesives (www.henkel-adhesives.com).

For preparing the spacer with laser cut capillaries, the spacer (MELINEX®329, PET film, thickness 250 μm) was coated with double sided adhesive tape on both sides and then capillaries (dimension: 90 mm×1.5 mm×0.28 mm) were cut with the laser MicroLine 6000 P (www.lpkf.com) at a frequency of 30 kHz and 3 W. The second laser-cutting step was used to generate the sample inlet and outlet in the cover foil. The spacer with laser cut capillaries and cover foil were then stick together as depicted in the ESM and finally joined on top of the substrate, orthogonal to the streptavidin multilayer, to get the ready to use lateral flow immunoassay, i.e. a microfluidic sensor.

The fluidic immobilization channel (dimension: 90 mm×1.5 mm×0.28 mm), initially used for the deposition of the PEM in 2.3 was similarly prepared with the exception that it only consisted of the spacer and the cover foil.

### Example 3: Biofunctionality of PPPs

A customized fluid control equipment and software from by Roche Diagnostics GmbH (Mannheim, Germany) were used. The fluid vent control was mounted on top of the outlet port.

To investigate the biofunctionality of polystreptavidin within the prepared PPPs in the multilayer, the streptavidin containing PEM was tested with the biotinylated Rhodamine 6G (Rh-6G, λₑₓ=525 nm, λₑₘ=548). Rh-6G was prepared in a 0.05 mg*mL⁻¹ stock solution with distilled water. 20 μL Rh-6G solution were injected into the LFA of the microfluidic sensor of Example 2 and were actively transported over the streptavidin containing PEM for 5 minutes. The unbound solution was removed and the streptavidin containing PEM was then analyzed with the fluorescence microscopy.

### PDADMAC:PSA Ratio and pH

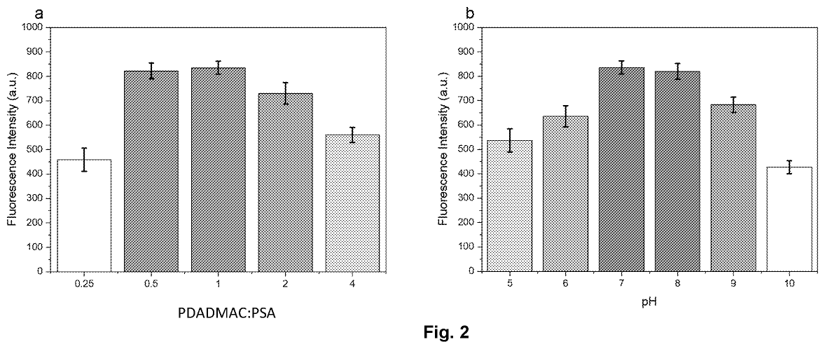

Binding of the dye by the polystreptavidin was investigated depending on the PDADMAC:PSA ratio and pH used for the preparation and application of the PPPs (FIG. 2). The lowest fluorescence signals were found at low and high PDADMAC concentrations (FIG. 2a). At low PDADMAC concentrations large PPPs with low positive charges were generated which represented a loose network. It was assumed that this may encourage streptavidin leaching as it was insufficiently bound by PDADMAC. In addition, the particles only showed a slightly positive charge, leading to a decrease of electrostatic interaction with PAA in the multilayer deposition. On the other hand, when PDADMAC was used in excess, a competition between PDADMAC itself and PPPs for the electrostatic binding sites with PAA in the multilayer formation resulted in less amount of streptavidin in the multilayer. Overall, the maximum fluorescence intensity was found at PDADMAC:PSA ratios of 0.5 and 1 suggesting that the highest amount of polystreptavidin could be securely immobilized within the multilayer. Due to economic reasons, the PDADMAC:PSA ratio of 0.5 was used for further experiments.

The pH of the PPP solution had a major effect on the ionization of the materials used (FIG. 2b). Whereas PDADMAC, as a strong cationic polyelectrolyte, has a positive charge over a wide pH range (pH 2-13) and is stable to pH shifts, the amphoretic polystreptavidin with the isoelectric point (IEP) of 5-6 is notably influenced by the pH of its environment. To ensure complexation of PDADMAC and pSA, both materials were mixed in different ratios at pH 7.4 and 150 mM NaCl. This pH level is slightly higher than pSA's isoelectric point (IEP), ensuring the overall negative charge of pSA, which in turn facilitates the electrostatic complexation with PDADMAC. Contrary, its neutral charge at pH 5 and 6 resulted in lower signals. This is supported by the known complexation process which is driven largely by a combination of electrostatic attraction between the constituents and entropy gains from counterion release. Basic pH values of 9 and 10 may have led to stronger interactions between polystreptavidin and PDADMAC but are of no consequence for the envisioned application of the assay. Considering the physiological environment of the envisioned assay, pH of 7.4 and ionic strength of 150 mM NaCl were hence kept constant in all subsequent experiments.

### Example 4: Performance of the Bioassay

A sandwich immunoassay with both buffer and plasma samples was carried out to investigate the assay performance of the LFA of Example 2, coupled to an external pump as described in Example 3. The analyte target was N-terminal prohormone of brain natriuretic peptide (NT-proBNP), which is a biomarker for heart failure. NT-proBNP biomarker samples were prepared in HBS buffer and human plasma through dilution of a stock solution to produce concentrations of 7.5, 15, 30, 60, 125, 250, 500, 1000, 2000, 4000, 6000 and 9000 pg*mL⁻¹, for analysis. The immunoassay was performed at room temperature (25° C.). The limit of detection (LOD) was calculated based on equitation (I) with the logistic fit parameter for the lower curve asymptote A and the standard deviation of the blind SD(blind):

LOD=A+3.3*SD(blind) (I)

### 4.1 Number of Layers

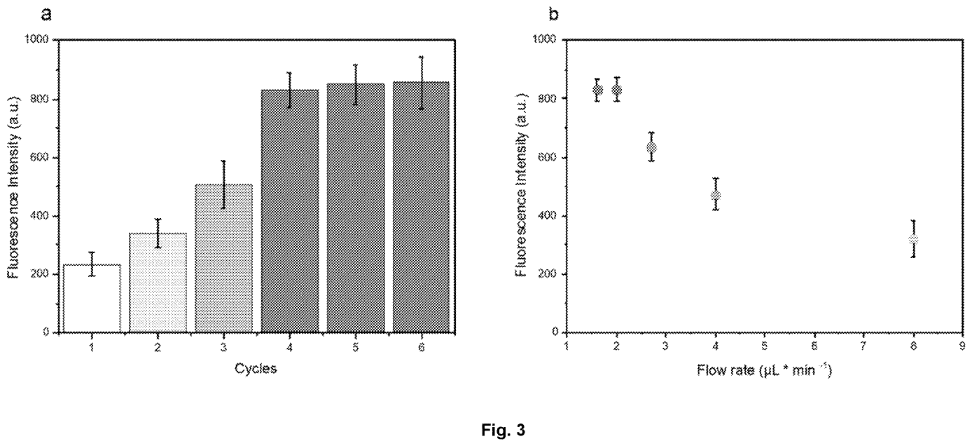

First, the number of layers was investigated using the sandwich assay of biotinylated capture antibodies (cAb), fluorescent reporter antibodies (Ab-fluorescence NPs) and NT-proBNP as analyte. cAbs were the capture antibodies (sheep anti-human NT-proBNP antibodies) from the 5th generation hs-BNP assay in the Elecsys®/cobas E™ platform. The Ab-fluorescence NPs comprised anti-BNP IgG detection antibodies (mouse anti-human NT-proBNP antibodies) targeting identical epitopes to their Elecsys®/cobas E™ analogue. The label were latex particles (Thermo Fisher Scientific), with a diameter of approx. 200 nm, which were dyed with squaraine (excitation/emission wavelengths: 652/665 nm) and coupled to the anti-BNP IgG detection antibody using EDC and sulfo-NHS. The results are graphically shown in FIG. 3.

It was found that fluorescence signals increased significantly with the first 8 layers (4 layers comprising polyanions, four layers comprising polycations), which suggests that the immobilized PSA concentration increased initially with the number of layers (FIG. 3a). Additional layers did not lead to further signal increase.

### 4.2 Flow Rate

Secondly, the effect of the flow rate on the assay was determined (FIG. 3b). Since the flow rate will also have an effect on the reconstitution of dried reagents, biotinylated cAbs and Ab-fluorescence NPs were first dried on the PET substrate. The 20 μL sample volume was transported with a flow rate of 8, 4, 2.7, 2 and 1.6 μL*min⁻¹. It was found that high flow rates resulted in low fluorescence signals (FIG. 2b) most likely due to less efficient capturing of the immune sandwich. In contrast, at flow rates ≤2 μL*min⁻¹ high fluorescence signals were obtained reaching a plateau. Therefore, a flow rate of 2 μL*min⁻¹ was chosen for further experiments.

4.3 Sandwich Immunoassay with Nanoparticles
4.3.1 Fluorescence Nanoparticles (dAb-Fluorescence NP)

The LFA of Example 2, wherein the human specific monoclonal anti-cardiac NT-proBNP detection antibody was modified with fluorescence nanoparticles (dAb-fluorescence NP), was used. The label were latex particles (Thermo Fisher Scientific), with a diameter of approx. 200 nm, which were dyed with squaraine (excitation/emission wavelengths: 652/665 nm) and coupled to the human specific monoclonal anti-cardiac NT-proBNP detection antibody using EDC and sulfo-NHS.

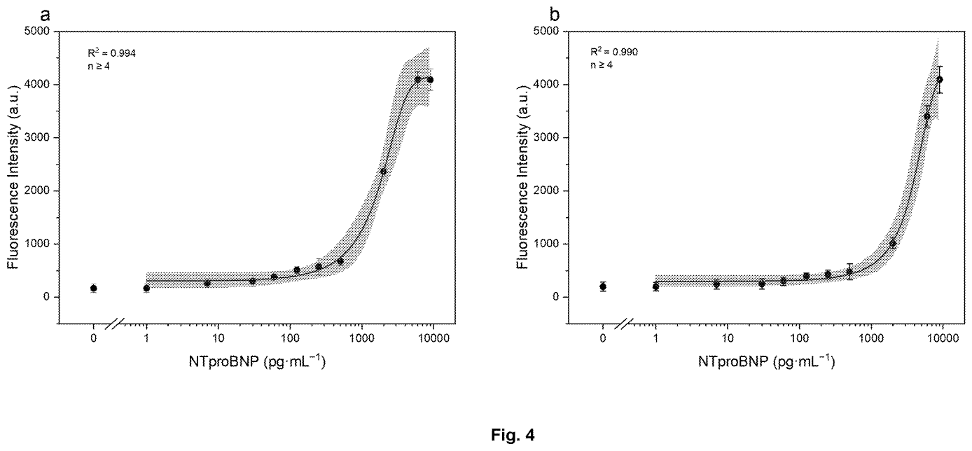

NT-proBNP-enriched buffer, plasma and whole blood were added to the LFA of Example 1 having 8 layers (4 layers comprising polyanions, four layers comprising polycations) at different concentrations and the fluorescence signals were recorded with a fluorescence camera. The raw images were analyzed with a Roche internal program that converts the detection zone pixels into brightness values from 0 to 4096. This allowed a concentration-dependent NT-proBNP evaluation of the generated fluorescence signals. The fluorescence signals were then plotted versus NT-proBNP concentration (logarithmic scale) and given a 95% confidence interval. The dAb-fluorescence NP has a mean size of 200 nm, an excitation wavelength of λex=652 nm, and an emission wavelength of λem=665 nm. No false positive signals were generated. The results are graphically shown in FIG. 4.

TABLE 1
Experimental Details.

| | |
|---|---|
| NT-proBNP concentration | 7-6000 pg/mL |
| Capture antibody | pAk<proBNP>S-IgG(IS,39-50)-Bi(X-OSu) |
| Detection antibody | L03-17-B03 |
| Sample volume | 20 μL |
| Sample matrix | HEPES Buffer, human whole blood, plasma |
| Reaction time | 20 min |
| Flow rate | 2 μL*min⁻¹ |

Based on the experimental results and using equitation (I), an LOD of 26.7 pg*mL-1 was calculated for buffer (n=6) and an LOD of 56.1 pg*ml-1 was calculated for plasma (n=6).

For comparison: The currently available cobas h 232 NT-proBNP Plus Test is reported by Roche to have a limit of detection (LOD) of 60 pg*mL⁻¹. From the above, it can be seen that the detection limit of the inventive microfluidic sensor had an equivalent or better LOD compared to the currently available cobas h 232 NT-proBNP Plus Test.

4.3.2 with Gold Nanoparticles (dAb-AuNP)

The LFA of Example 2, wherein the human specific monoclonal anti-cardiac NT-proBNP detection antibody was modified with gold nanoparticles (dAb-AuNP), was used as described in 4.3.1.

NT-proBNP-enriched plasma was added to the LFA at different concentrations and the test signal from the gold nanoparticles was recorded with a camera. The raw images were analyzed with Image J, which converts the detection zone pixels into gray values. This allowed a concentration-dependent NT-proBNP evaluation of the generated signals. No false positive signals were generated.

For comparison: The currently available cobas h 232 NT-proBNP Plus Test is specified by Roche with a limit of detection (LOD) of 60 pg*mL⁻¹ and requires a sample volume of 150 μL. Based on experimental results and using equitation (I), an LOD of 500 pg*mL⁻¹ was calculated for plasma (n=6) with only 12 μL sample volume.

TABLE 2
Experimental details.

| | |
|---|---|
| NT-proBNP concentration | 500-6000 pg/mL |
| Capture antibody | pAk<proBNP>S-IgG(IS,39-50)-Bi(X-OSu) |
| Detection antibody | MAK<proBNP>M-18.4.34-IgG-Gold |
| Sample volume | 12 μL |
| Sample matrix | human plasma |
| Reaction time | 20 min |
| Flow rate | 0.6 μL*min⁻¹ |

### Example 5: Storage Stability

The overall stability of the assembled LFA of Example 2 was investigated. While the antibodies and polystyrene Ab-fluorescence NPs are known to be stable when stored in dry stage through a fleece-based system, their storage within a non-fleece, i.e. membrane free, Point-of-Care-Testing (POCT) was not yet known. The capture antibodies and polystyrene Ab-fluorescence NPs were dried in a sucrose matrix supporting the stability maintenance of both reagents in the dried state according to Starciuc et al. (T. Starciuc et al., “Trehalose or Sucrose: Which of the Two Should be Used for Stabilizing Proteins in the Solid State? A Dilemma Investigated by In Situ Micro-Raman and Dielectric Relaxation Spectroscopies During and After Freeze-Drying,” Journal of pharmaceutical sciences, vol. 109, no. 1, pp. 496-504, 2020, doi: 10.1016/j.xphs.2019.10.055).

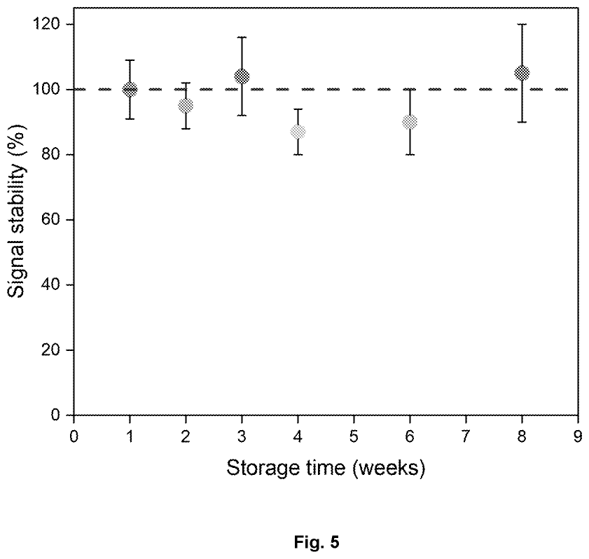

Over a period of 8 weeks, no loss in activity could be observed. It was assumed that complexation of streptavidin and the incorporation of the PPPs in a multilayer also contributed to stable and reproducible signal values. This effect was linked to the additional hydrated state of the assemble, having a protective effect on the immobilized streptavidin. The results are graphically shown in FIG. 5.

1. A detection zone of a lateral flow immunoassay device comprising a polyion multilayer, the polyion multilayer comprising
(i) at least one layer comprising a complex of at least a first polyion and a first member of a binding pair; and
(ii) at least one layer comprising a second polyion, which is oppositely charged with respect to the first polyion;
wherein the detection zone of the lateral flow immunoassay device is membrane-free.

2. The detection zone of a lateral flow immunoassay device of claim 1, wherein the first polyion of (i) and the second polyion of (ii) are selected from the group consisting of acrylamide copolymer, alginate, lignosulfonate, pectin, polyacrylic acid, co-polymer of polyacrylic acid, polyvinylsulfuric acid, polycarboxylic acid, polyphosphoric acid, polysaccharide, crosslinked polystyrene sulfonic acid, uncrosslinked polystyrene sulfonate, poly-diallyldialkyl-ammonium chloride, polyethyleneimine, polyvinylamine, polyvinylpyridine, polyvinylammonium chloride, partial or complete salts of these polyions, wherein the counter ions are selected from alkali cations, halogenide anions, and sulfonate, and mixtures of two or more of these polyions and/or their partial or complete salts;
with the condition that first and second polyion are selected so that they have opposite charges.

3. The detection zone of a lateral flow immunoassay device of claim 1, wherein the first polyion of (i) and the second polyion of (ii) are selected from the group consisting of poly(styrenesulfonate) (PSS), poly(vinyl sulfonic acid, (PVS), polyacrylic acid (PAA), poly(acrylamide-2-methylpropane sulfonate) (PAMPS), dextran sulfate, polyethylenimine (PEI), poly(diallyldimethylammonium chloride) (PDADMAC), poly(vinylamine) (PVA), polyallylamine (PAH), poly(vinylbenzyltrimethyl ammonium) (PVTA), poly(N,N-dimethyl-3,5-dimethylene piperidinium) (PDDP), poly-L-lysine (PLL), chitosan, and mixtures of two or more thereof, with the condition that first and second polyion are selected so that they have opposite charges.

4. The detection zone of a lateral flow immunoassay device of claim 1, wherein the first polyion of (i) comprises one or more polycation(s), wherein the one or more polycation(s) of (i) are selected from the group consisting of PEI, PDADMAC, PAH, PLL, chitosan, and mixtures of two or more thereof.

5. The detection zone of a lateral flow immunoassay device of claim 1, wherein the second polyion of (ii) comprises one or more polyanion(s), wherein the one or more polyanion(s) of (ii) are selected from the group consisting of PSS, PVS, PAA, dextran sulfate, and mixtures of two or more thereof.

6. The detection zone of a lateral flow immunoassay device of claim 1, wherein the first member of a binding pair is selected from the group consisting of antibody, nanoparticle functionalized with an antibody, nanoparticle functionalized with streptavidin, enzyme, (poly)streptavidin, biotin, complementary nucleic acid, protein A, protein G, molecular imprinted polymer (MIP), and mixtures of two or more thereof, wherein optionally the first member of a binding pair of (i) is streptavidin, polystreptavidin, or negatively charged polystreptavidin.

7. The detection zone of a lateral flow immunoassay device of claim 1, wherein the polyion multilayer is positioned on a surface of a carrier, wherein optionally the carrier is a foil, optionally selected from the group consisting of gold foil, silver foil, carbon foil and polymer foil, wherein the polymer is selected from the group consisting of polyester, polyvinyl chloride, poly(methyl methacrylate), polydimethylsiloxane and mixtures of two or more of these polymers, wherein the polymer foil is optionally a polyester foil, or a poly(ethylene terephthalate) (PET) foil.

8. The detection zone of a lateral flow immunoassay device of claim 1, wherein in the polyion multilayer, the layers are arranged horizontally on top of each other with respect to the carrier, wherein optionally a layer comprising the second polyion (ii) is horizontally positioned on top of a layer comprising the first polyion and the first member of a binding pair (i); wherein optionally, the polyion multilayer comprises at least two layers (i) and at least two layers (ii), wherein the layers (i) and (ii) are horizontally positioned in an alternating sequence on top of each other; wherein optionally, the final layer on top comprises one or more polyanion(s).

9. A lateral flow immunoassay device comprising a capillary channel, wherein the capillary channel houses
(I) a detection zone as defined in claim 1;
(II) a first reagent zone, which comprises a labeled binding moiety; and
(III) a second reagent zone, which comprises a binding moiety carrying a second member of a binding pair;
wherein the binding moieties of (II) and of (III) are both capable of binding an analyte of interest, and the second member of a binding pair is capable of binding with the first member of a binding pair of the detection zone;
wherein first reagent zone of (II) and second reagent zone of (III) are spatially separated or overlap with each other at least partially.

10. The lateral flow immunoassay device of claim 9, further comprising an inlet opening configured to receive a sample of a body fluid; a vent opening configured to provide an air vent to the capillary channel; and at least one measuring device.

11. A method for determining an analyte in a sample, the method comprising
(a) contacting a sample with at least a labeled binding moiety and a binding moiety carrying a second member of a binding pair, thereby forming a mixture;
(b) contacting the mixture formed in (a) with a polyion multilayer, the polyion multilayer comprising
(i) at least one layer comprising a complex of at least a first polyion and a first member of a binding pair,
(ii) at least one layer comprising a second polyion,
thereby optionally forming complexes;
(c) determining the amount of label containing complexes formed in (b);
(d) determining said analyte in a sample based on the result of step (c).

12. The method of claim 11, wherein the sample is a body fluid sample of a subject, selected from the group consisting of saliva, urine, nasal fluid, blood, plasma and serum sample of a subject.

13. A kit for determining an analyte in a sample, comprising the lateral flow immunoassay device of claim 9 and a pump, which is connected or connectable to the suction device.
